(19) United States
(12) Patent Application Publication
(10) Pub. No.: US 2012/0302927 A1
(43) Pub. Date: Nov. 29, 2012

(54) METHODS FOR CHARACTERIZING NONLINEAR FIELDS OF A HIGH-INTENSITY FOCUSED ULTRASOUND SOURCE AND ASSOCIATED SYSTEMS AND DEVICES

(75) Inventors: Vera A. Khokhlova, Seattle, WA (US); Petr Yuldashev, Moscow (RU); Oleg A. Sapozhnikov, Seattle, WA (US); Michael R. Bailey, Seattle, WA (US); Lawrence A. Crum, Bellevue, WA (US)

(73) Assignee: University of Washington, Seattle, WA (US)

(21) Appl. No.: 13/479,067

(22) Filed: May 23, 2012

Related U.S. Application Data
(60) Provisional application No. 61/488,998, filed on May 23, 2011.

(51) Int. Cl. A61N 7/00

(52) U.S. Cl. 601/2

(57) ABSTRACT

The present technology is directed to methods for characterizing nonlinear ultrasound fields and associated systems and devices. In several embodiments, for example, a method of calculating output of a high intensity focused ultrasound (HIFU) device comprises treating a target site with a multi-element HIFU array. In some embodiments, the array comprises a generally spherical segment. The method can further include simulating a field of the array by setting a boundary condition for the array. The boundary condition can be set by simplifying at least one geometrical aspect of the generally spherical segment.

### CROSS-REFERENCE TO RELATED APPLICATION(S)

This application claims the benefit of pending U.S. Provisional Application No. 61/488,998, filed May 23, 2011, which is incorporated herein by reference in its entirety.

### FEDERAL FUNDING STATEMENT

This invention was made with government support under EB007643, awarded by National Institutes of Health (NIH), and under SMST001601, awarded by National Space Biomedical Research Institute (NSBRI). The government has certain rights in the invention.

### TECHNICAL FIELD

The present technology relates generally to high intensity focused ultrasound. In particular, several embodiments are directed toward methods and systems for non-invasive treatment of tissue using high intensity focused ultrasound therapy.

### BACKGROUND

Minimally invasive and non-invasive therapeutic ultrasound treatments can be used to ablate, necrotize, and/or otherwise damage tissue. High intensity focused ultrasound (“HIFU”), for example, is used to thermally or mechanically damage tissue. HIFU thermal treatments increase the temperature of tissue at a focal region such that the tissue quickly forms a thermally coagulated treatment volume. HIFU treatments can also cause mechanical disruption of tissue with well-demarcated regions of mechanically emulsified treatment volumes that have little remaining cellular integrity.

A current trend in HIFU medical technologies is to use two-dimensional multi-element phased arrays with the elements distributed over a segment of a spherical surface. Each element of such an array is controlled independently, which makes it possible to electronically steer the focus in space, to create a complex field configuration in the form of several foci, and to minimize the heating of acoustic obstacles (for instance, ribs) while maintaining high intensities at the focus. The arrays can also be utilized to improve the quality of focusing in inhomogeneous tissue using time reversal methods, as well as to trace the region of treatment, which shifts due to respiration.

In many HIFU applications, the acoustic intensity in situ can reach several tens of thousands of watts per square centimeter (W/cm²), causing nonlinear propagation effects. Nonlinear effects can result in formation of weak shocks in the ultrasound waveform, which fundamentally change the efficiency of ultrasound thermal action on tissue, and can lead to new biological effects of a non-thermal nature. However, measurement of all the permutations of an array in water is time consuming and difficult to extrapolate to tissue. Numerical experimentation is an important tool in characterizing pressure fields created by HIFU radiators, in developing exposure protocols, and in predicting corresponding HIFU-induced biological effects in tissue. Simulations work for both water and tissue, but full 3D nonlinear modeling is difficult and computationally expensive. Therefore, there is a need to create reliable and effective methods to characterize three-dimensional fields of multi-element HIFU arrays and properly account for the formation of shocks.

### BRIEF DESCRIPTION OF THE DRAWINGS

FIG. 1 is a schematic view of a HIFU system configured in accordance with an embodiment of the present technology.

FIG. 2 is a block diagram illustrating a method of modeling three-dimensional multi element HIFU arrays in accordance with an embodiment of the present technology.

FIGS. 3A-3C are geometric models of acoustic fields radiated by a multi-element HIFU array using various boundary conditions in accordance with an embodiment of the present technology.

FIGS. 4A-4C are front views of boundary conditions in the initial plane for the array models shown in FIGS. 3A-3C, respectively.

FIGS. 5A and 5B are graphs comparing pressure distributions of a HIFU array and simplified HIFU radiation models in accordance with an embodiment of the present technology.

FIG. 6 is a series of illustrations comparing pressure waveforms in the focus of a HIFU array with simplified HIFU equivalent source models at various intensities in accordance with an embodiment of the present technology.

FIG. 7 is a series of pressure distributions comparing peak pressure amplitudes of a HIFU array with simplified HIFU equivalent source models at various intensities in accordance with an embodiment of the present technology.

### DETAILED DESCRIPTION

The present technology is directed to methods for characterizing nonlinear ultrasound fields and associated systems and devices. In several embodiments, for example, a method of calculating output of a HIFU device comprises treating a target site with a HIFU array having nonlinear propagation effects. In some embodiments, the array comprises a generally spherical segment. The method can further include simulating a field of the array by setting a boundary condition for the array. Setting a boundary condition can include simplifying at least one geometrical aspect of the generally spherical segment (e.g., modeling a multi-element spherical array as a single-element flat transducer). By modeling the nonlinear effects using the simplified boundary condition, effects of the HIFU treatment parameters can be more readily discerned.

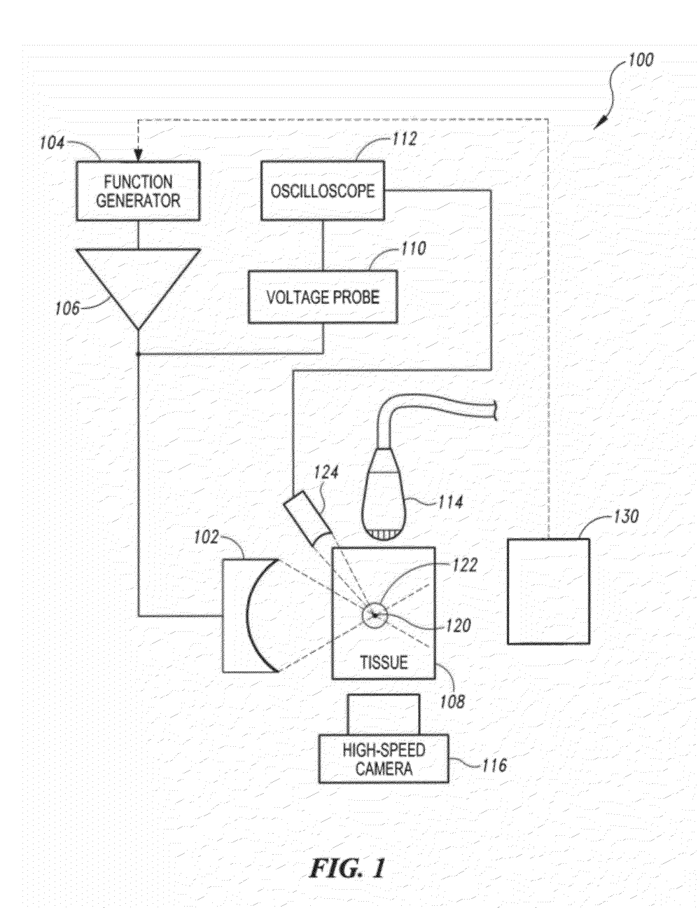

Certain specific details are set forth in the following description and in FIGS. 1-7 to provide a thorough understanding of various embodiments of the technology. For example, several embodiments of HIFU treatments that destroy tissue are described in detail below. The present technology, however, may be used to destroy multi-cell structures similar to tissue. Additionally, the term “target site” is used broadly throughout the disclosure to refer to any volume or region of tissue that may benefit from HIFU treatment. Other details describing well-known structures and systems often associated with ultrasound systems and associated devices have not been set forth in the following disclosure to avoid unnecessarily obscuring the description of the various embodiments of the technology. A person of ordinary skill in the art, therefore, will accordingly understand that the technology may have other embodiments with additional elements, or the technology may have other embodiments without several of the features shown and described below with reference to FIGS. 1-7.

### I. HIFU SYSTEMS

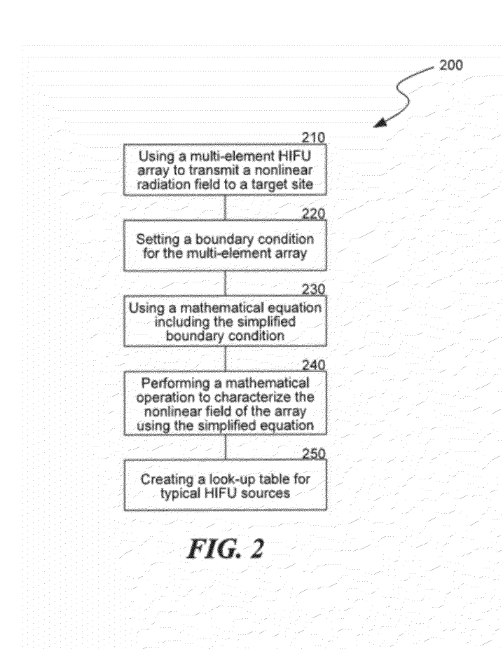

FIG. 1 is a schematic view of a HIFU system 100 configured in accordance with an embodiment of the present technology. The HIFU system 100 can include a HIFU source 102 operably coupled to a function generator 104 and an amplifier 106. The HIFU source 102 can be an ultrasound transducer that emits high levels of ultrasound energy to a focus 120. The focus 120 can be a point, plane, or region at which the intensity from the HIFU source 102 is the highest. In some embodiments, for example, the HIFU source comprises a generally spherical, 256 element array. In other embodiments, the array can have other shapes or number of elements. As will be described in further detail below beginning with FIG. 2, the multi-element array can induce complex, nonlinear effects in tissue.

Referring back to FIG. 1, in one embodiment the HIFU source 102 can have a frequency range of approximately 0.5-20 MHz. In other embodiments, however, the frequency of the HIFU source 102 can vary. The function generator 104 (e.g., an Agilent 33250A function generator from Agilent Technologies, Inc. of Santa Clara, Calif.) and the amplifier 106 (e.g., an ENI A-300 300 W RF amplifier from Electronic Navigation Industries (ENI) of Rochester, N.Y.) can drive the HIFU source 102 to generate pulsed shock waves proximate to the focus 120. Accordingly, the HIFU system 100 can implement a pulsing protocol in which ultrasound frequency, pulse repetition frequency, pulse length, duty cycle, pressure amplitude, and/or other factors associated with the HIFU treatment can be adjusted to generate shock waves proximate to the focus 120.

During treatment, the HIFU source 102 can be positioned proximate to tissue 108, and the focus 120 of the HIFU source 102 can be aligned with at least a portion of a target site 122 within the tissue 108. For example, the HIFU source 102 can be positioned over a patient's kidney, heart, or liver, and the focus 120 can be aligned with infected or otherwise adverse tissue therein. In still other embodiments, a variety of other types of tissue may be treated using the HIFU system 100. Larger target sites 122 can be mechanically fractionated by scanning the HIFU source 102 over the treatment region using either mechanical or electronic scanning. Such scanning and the initial positioning of the HIFU source 102 can be performed manually or mechanically (e.g., using a three-axis positioning system, not shown). The function generator 104 can initiate the pulsing protocol to generate shock waves with amplitudes between approximately 10 MPa and approximately 100 MPa at the focus 120 with the HIFU source 102 having a frequency of approximately 2 MHz. In other embodiments, such as at lower or higher ultrasound frequencies, the shock wave amplitudes of the HIFU source 102 can be greater or smaller. Absorption of ultrasonic energy occurs primarily at the shock front and induces heating of the tissue 108 that can exceed boiling temperature in the tissue 108.

During each HIFU pulse, one or more boiling bubbles can be formed in the tissue. The superheated vapor of the boiling bubbles provides a force pushing outward from the bubble. This repetitive explosive boiling activity and interaction of the ultrasound shock waves with the boiling bubbles emulsifies the tissue 108 at the target site 122 to form a liquid-filled lesion, at least partially devoid of cellular structure, with little to no thermal coagulation within the treated region. The reflection of the shock wave from the surface of these millimeter-sized boiling bubbles can also form cavitation bubbles proximate to the boiling bubble that can also induce mechanical damage to tissue.

The HIFU system 100 can also include systems or devices that detect and monitor tissue ablation initiation and the activity (e.g., heating or bubble activity) in the tissue 108. In the embodiment illustrated in FIG. 1, for example, the HIFU source 102 is operably coupled to a voltage probe 110 and an oscilloscope 112 that can monitor and record, respectively, the drive voltage at the HIFU source 102. In other embodiments, however, the HIFU source 102 may be coupled to additional detection and/or monitoring devices.

The HIFU system 100 can also include a passive cavitation detector (“PCD”) 124 that monitors acoustic signals associated with tissue ablation. For example, the PCD 124 can include an acoustic receiver (e.g., an ultrasound transducer) separate from the HIFU source 102, but confocally aligned with the focus 120 of the HIFU source 102 such that the PCD 124 can receive real-time acoustic feedback during HIFU treatment. As shown in FIG. 1, similar to the voltage probe 110, the PCD 124 can also be coupled to the oscilloscope 112 to record acoustic signals during HIFU treatment.

Echogenic ablation activity and/or the thermal effects of the HIFU treatment can also be monitored using separate devices and systems. The HIFU system 100 illustrated in FIG. 1, for example, includes an imaging system 114 that can create a visual image to monitor the boiling bubbles and thus temperatures of approximately 100° C. in real-time at a depth within the tissue 108. The imaging system 114 can be a separate confocal transducer, an unfocused transducer, another type of confocal or unfocused ultrasound source, one or more sub-element(s) of a multi-element array, and/or a separate imaging system. For example, in one embodiment the imaging system 114 includes an HD1-1000 scanner with a CL 10-5 scanhead made by Philips Medical Systems of Bothell, Wash. In other embodiments, the imaging system 114 can include a magnetic resonance imaging (“MRI”) system that can monitor temperature and boiling activity during HIFU treatments or other suitable devices.

In the embodiment shown in FIG. 1, the HIFU system 100 also includes a high-speed camera 116 (e.g., video, still frame) to take video or still images of the target site 122 during HIFU treatment to capture the effects of the HIFU treatment on the tissue 108. Such a camera 116 is generally used with initially transparent tissues or tissue phantoms to capture the thermal effects of HIFU treatment within the tissue 108. Accordingly, the high-speed camera 116 can be especially suited for experiments and testing that include transparent gel phantoms to simulate tissue. The high-speed camera 116 is an optional component that may not be used in some embodiments.

The HIFU system 100 can also simulate the shock waves and heating in water or tissue. Resultant modeling can be used to calculate heating from the shock amplitude of the focal waveform, and for extrapolating pressure waveforms at the focus 120 in water to the equivalent waveforms in tissue. One such method for this extrapolation is called “derating,” and is useful for regulatoty oversight and HIFU treatment planning. For example, derating can be used to determine values of the nonlinear acoustic field parameters in the tissue region exposed to HIFU (e.g., the target site 122 and the surrounding tissue 108). During the nonlinear derating process, pressure waveforms are measured and/or modeled in water at the focus 120 at various source outputs. The source outputs are then scaled to generate the same focal waveform with the same focal pressure and focal shape in tissue.

The HIFU system 100 can also include a testing apparatus 130 that can assess the extent of mechanical and/or thermal ablation and distinguish among lesion types. In some embodiments, for example, the testing apparatus 130 can send feedback to the function generator 104 or other components of the HIFU system 100 to cause the function generator 104 to select ultrasound parameters designed to achieve a particular type of mechanical or thermal ablation. In other embodiments, the HIFU system 100 can include a different arrangement and/or may not include a number of features recited above.

### II. MODELING MULTI-ELEMENT ARRAYS

The present technology includes systems and methods for simulating nonlinear effects in a focal region of a multi-element array based on a simplified model (an “equivalent source”) with a single-element boundary condition. FIG. 2 is a block diagram illustrating a method 200 of modeling three-dimensional multi-element HIFU arrays in this manner. The method 200 can be used with the HIFU system 100 of FIG. 1 or other suitable systems. The method 200 includes using a multi-element HIFU array to transmit a nonlinear radiation field to a target site. (Block 210) The method 200 further includes setting a boundary condition for the multi-element array. (Block 220) By setting a boundary condition, at least one geometric aspect of the radiation segment is simplified. In some embodiments, for example, a multi-element, generally spherical array segment is modeled as a simpler geometric shape (e.g., a single-element, generally circular shape having a flat plane with a generally uniform distribution of the amplitude and parabolic phase substituted for the spherical array). The method 200 further includes using a mathematical equation including the simplified boundary condition. (Block 230) As will be discussed in detail below, a simplified, two-dimensional boundary condition can be substituted for a more complex three-dimensional array in various modeling equations. The method 200 further includes performing a mathematical operation to characterize the nonlinear field of the array using the simplified equation. (Block 240) By simplifying the geometry of the array segment, the nonlinear effects of the radiation can be more readily determined. In several embodiments, the nonlinear effects are first determined in a testing environment (e.g., in water). The resulting field characterization can then be extrapolated and applied to characterize HIFU treatment effects in situ. In further embodiments, alternate or additional aspects of the modeling equation can be simplified. In some embodiments, the method can additionally include creating a look-up table for typical HIFU sources. (Block 250) The look-up table can be used, for example, to characterize and value nonlinear effects from a given source.

As will be described in further detail below, the mathematical operations performed on the simplified model can take on various forms in different embodiments of the technology. For example, a “Westervelt model” can include substituting a single, uniformly vibrating (in terms of the pressure and magnitude), spherical element for the array component in the Westervelt equation, thereby decreasing the dimensions of the equation from three-dimensional in spatial coordinates to two-dimensional (axially symmetric). In another embodiment, a Khokhlov-Zabolotskaya-Kuznetsov (“KZK”) model can include substituting a single, uniformly vibrating, flat element (e.g., a single focused piston source) for the array element in the KZK equation. The effective dimensions are again decreased to two, and the KZK equation can be relatively easier/quicker to solve than the more complicated Westervelt equation.

A. The Westervelt Model

As discussed above, in some embodiments, the field of the array can be simulated according to the Westervelt equation, which in the accompanying system of coordinates can be written in the form

$$\frac{{\partial }^{2} p}{\partial τ \partial z} = \frac{{c}_{0}}{2} Δ p + \frac{β}{2 {ρ}_{0} {c}_{0}^{3}} \frac{{\partial }^{2} {p}^{2}}{\partial {τ}^{2}} + \frac{δ}{2 {c}_{0}^{3}} \frac{{\partial }^{3} p}{\partial {τ}^{3}} \qquad ( 1 )$$

Here, p is acoustic pressure, z is the spatial coordinate along the beam axis, τ=t−z/c₀, t is time, Δp=∂²p/∂z²+∂²p/∂y²+∂²p/∂x², x and y are spatial coordinates lateral to z; ρ₀, c₀, β, and δ are the density, ambient sound speed, nonlinearity coefficient, and absorption coefficient of the medium, respectively. Calculations can be performed for water, and the corresponding physical parameters in Eq. (1) can be as follows; ρ₀=1000 kg/m³, c₀=1500 m/s, β=3.5, and δ=4.33×10⁻⁶ m²/s. The origin of the coordinates corresponded to the center of a spherical segment where individual elements of the array were located so that the point x=0, y=0, z=F corresponded to the geometric focus of the array. Equation (1), which governs the propagation of nonlinear waves in a thermoviscous medium in the positive direction of the z axis, can be used to simulate weakly nonlinear and weakly focused fields generated by diagnostic ultrasound transducers.

To solve the Westervelt equation (1), written in the evolution form in terms of the z coordinate, it is necessary to assign boundary conditions on some initial plane (x, y, z=z₀). Since the elements of the array are distributed on the surface of a spherical cup, the field was first calculated on the plane z₀=2 cm from the center of the array using the Rayleigh integral. This plane is located near the edge of the array cup, which is at a distance of z=1.85 cm from the array center.

$$p ( \vec{r} ) = - i {ρ}_{0} {c}_{0} \frac{k}{2 π} {\int }_{S}^{ } \frac{u ( {\vec{r}}^{′} ) exp ( i k \vert \vec{r} - {\vec{r}}^{′} \vert )}{\vert \vec{r} - {\vec{r}}^{′} \vert} d {S}^{′} , \qquad ( 2 )$$

where k=ω/c₀ is the wavenumber, ω=2 πf, f is the ultrasound frequency, and u(\vec{r}′) is the complex amplitude of the vibration velocity of the radiator surface S. In other embodiments, the boundary condition is set by acoustic holography or other methods.

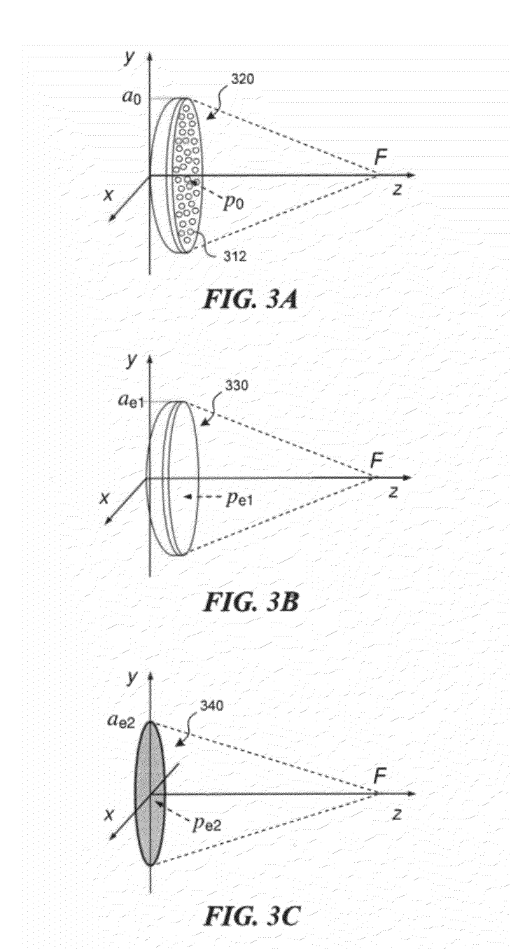

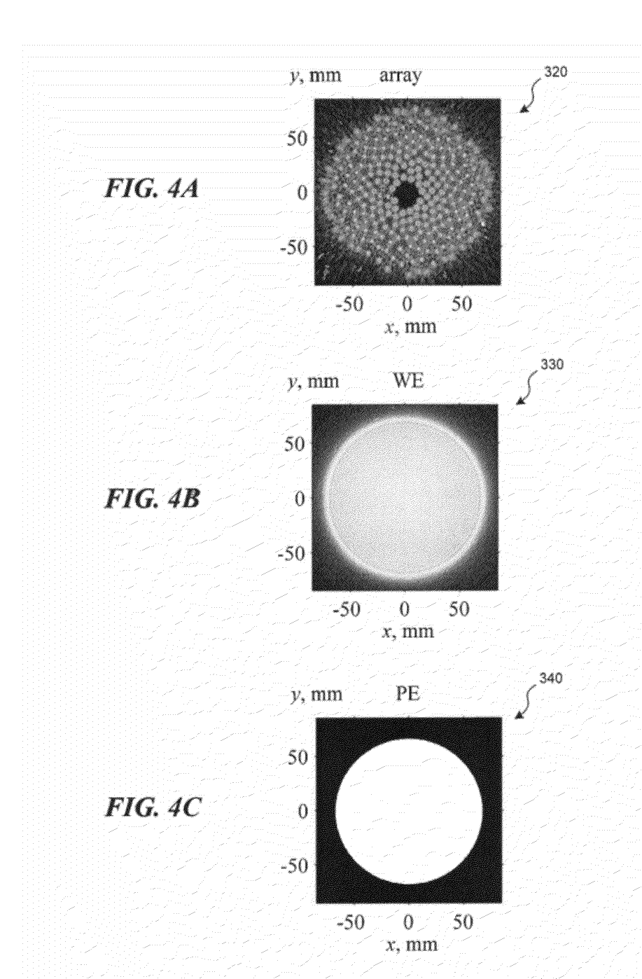

As discussed above, in several embodiments, multi-element three-dimensional HIFU arrays can induce complex, nonlinear effects in tissue. FIG. 3A, for example, illustrates a representative, generally spherical multi-element array 320. In this embodiment, individual elements 312 are positioned on the generally spherical cup with the radius of curvature F and aperture a₀. FIG. 4A illustrates a front view of a pressure distribution of the acoustic field of the array 320 translated to the plane z=0 using an angular spectrum method. The nonlinear effects of the array 320 can be simulated using a 3D full diffraction Westervelt-type equation. Such complete analysis can be complex and time-consuming.

To simplify the analysis of the nonlinear effects of the HIFU radiation, the array in the Westervelt equation is substituted by an equivalent single-element focused piston source 330, as illustrated in FIG. 3B. A boundary condition for acoustic pressure magnitude pₑ₁ is given on the spherical cup with radius of curvature F and aperture aₑ₁. FIG. 4B is a front view of the acoustic field of the single-element focused piston source 330 translated to the plane z=0. Using this substituted model, the three-dimensional field is characterized using two-dimensional spatial coordinates. The analysis thus becomes comparatively quicker and easier.

B. The KZK Model

FIG. 3C is a geometric model of a multi-element HIFU array using a single focused piston source 340 as a boundary condition, and FIG. 4C is a front view of the acoustic field of the single-focused piston source 340 translated to the plane z=0. Referring to FIGS. 3C and 4C, using the KZK model, the z-axis spatial coordinate is eliminated from the Westervelt equation, and the complex nonlinear effects can be modeled by using the axially-symmetric, parabolic KZK equation to simulate the acoustic field. The array in the KZK equation is substituted by the equivalent single-element piston source 340 with aperture aₑ₂. The boundary condition for acoustic pressure magnitude pₑ₂ is given in the plane z=0, and the parabolic phase distribution provides focusing at z=F. In this embodiment, the corresponding boundary condition in the three-dimensional algorithm was set at z=0 in the form of a round piston with phase k₀(x²+y²)/2F. In some embodiments, the KZK equation can be easier and faster to solve than the Westervelt equation.

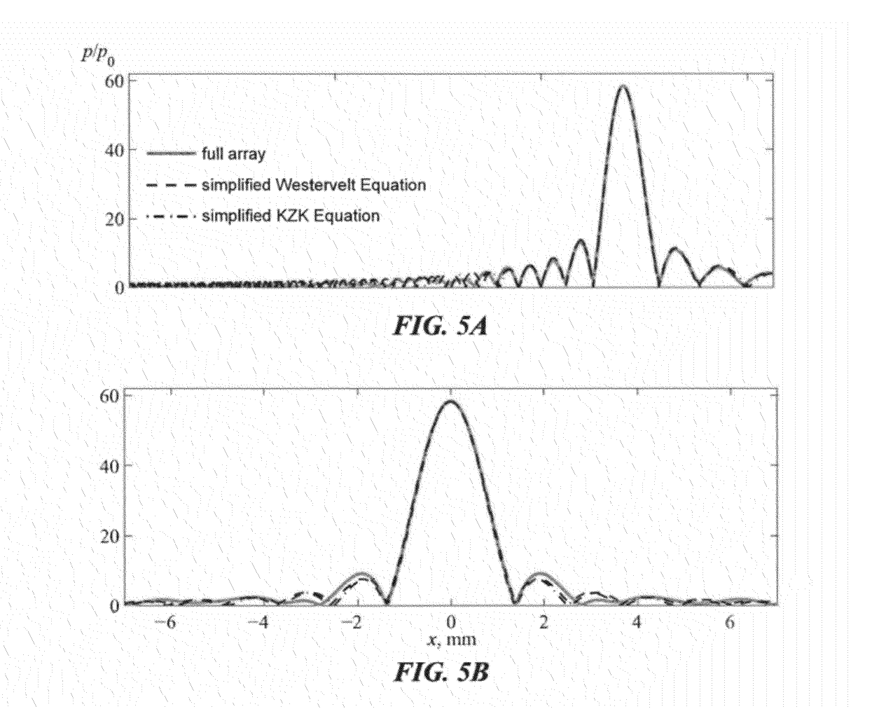

In both of the Westervelt and KZK models, at least two parameters must be determined for the single element models: effective aperture and initial pressure that corresponds to a certain output of the array. FIGS. 5A and 5B, for example, are graphs illustrating how to determine these parameters (aₑ₁, pₑ₁, aₑ₂, pₑ₂) of the equivalent sources. This determination is made by varying these two parameters and finding the best match to the linear (low output) pressure distribution on the axis of the array in the focal region (FIG. 5A) and off-axis in the focal plane (FIG. 5B). In other words, variation of the aperture of the single element changes the width of the focal lobe. Larger elements create narrower focal lobe both axially and radially in the focal plane. Once a width is found as the best match, the initial pressure determines the peak value of the pressure in the focus. When the initial pressure is increased, the degree of nonlinear effects in the focal region will be the same as for the array field when the pressure at the array elements correspondingly increases. The best match for both equivalent sources is shown in FIGS. 5A and 5B in dashed line.

In a further embodiment, the results of the KZK model can be used to create a data base or a look-up table with results for the cases that are within the range of typical HIFU sources. For example, the KZK equation can be rewritten in nondimensional form and all physical parameters of any equivalent source can be reduced to only two parameters: linear focusing gain (G) and proportion to the initial pressure amplitude (N). The KZK model can then be run in two parameter space for different G and N combinations, and the results can be stored in a database or look-up table, or presented as curves. A user can find the effective parameters N and G for their array transducer by measuring the axial field at low power and finding the effective aperture and amplitude. The aperture defines G. The low amplitude can be scaled back to the level of interest to find N. The look-up table can then provide information regarding the type and significance of nonlinear effects.

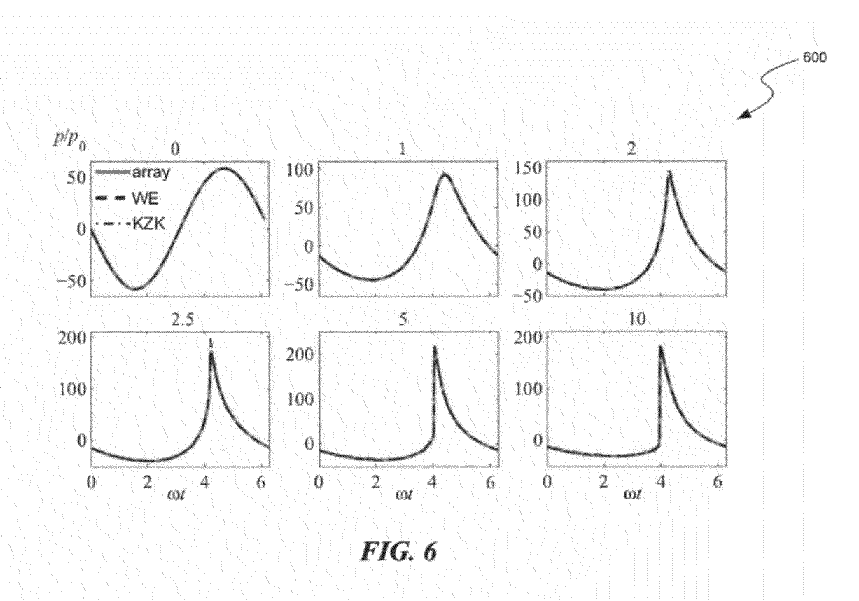

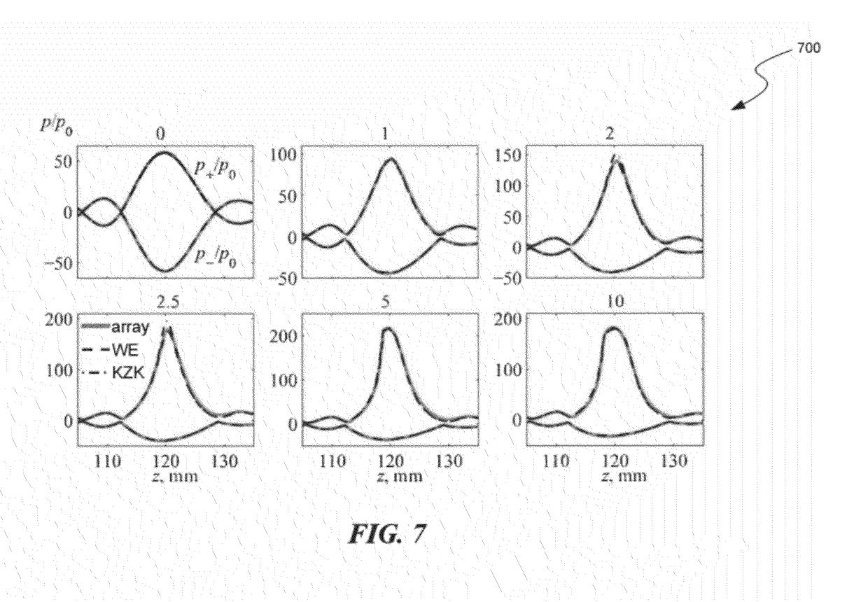

As will be discussed in further detail below with reference to FIGS. 6 and 7, waveform analysis has shown that the Westervelt and KZK simplified models are quite accurate in replicating a full, non-simplified analysis in the focal region of the array.

3. Validation

The accuracy of the numerical solutions obtained with the simplified equivalent source models may be examined by comparing the simulation results with known analytical solutions or numerical simulations performed using other methods. FIG. 6, for example, is a series of waveform illustrations 600 comparing pressure amplitudes of a HIFU array with simplified HIFU equivalent source models at various intensities. FIG. 7 is a series of waveform illustrations 700 comparing peak pressure amplitudes of a HIFU array with simplified HIFU equivalent source models at various intensities. The number on the top of each frame corresponds to the initial intensity at the array elements in W/cm². The initial pressure amplitude of the equivalent sources is scaled from the low output (linear) modeling proportionally to the pressure output of the array p₀. In FIG. 6, one cycle of the simulated pressure waveforms in the focus at z=F is shown for the array (shown in solid line) and for two equivalent sources (shown in dashed line for the Westervelt equation (WE) and KZK equation). In FIG. 7, the peak pressures along the z axis and in the focal plane off-axis obtained in modeling of the field of the array (shown in solid line) and the fields of equivalent sources (shown in dashed line for the Westervelt equation (WE) and KZK equation). As shown in both FIGS. 6 and 7, there is significant agreement among all three waveforms in the focus, the curves essentially coincide.

The modeling systems and methods described herein can offer several advantages over existing technology. For example, the equivalent source models are expected to make it possible to simulate three-dimensional nonlinear fields of focused ultrasound radiators including formation of shocks in the focal region. Test results have shown high accuracy of the developed models, particularly for the focal lobe and several protocol and postfocal lobes. It is further expected that the technology may be used to solve a broad class of practically important problems of nonlinear medical acoustics. For example, the disclosed technology may be used to perform nonlinear ultrasound characterization of pressure fields of ultrasound HIFU surgical devices in water and/or to calculate ultrasound-induced thermal effects in tissue. Generalization of the algorithm with account for smooth inhomogeneities in the propagation medium may enable more realistic simulations in soft tissues. It is also expected that the present technology will make it possible to model ultrasound exposures in tissue with the presence of acoustic obstacles, e.g., during irradiation through the rib cage. One feature of the algorithms described herein, for example, is the possibility of calculating three-dimensional fields of radiators with complex spatial configuration while maintaining reasonable requirements on the computing resources available.

### III. CONCLUSION

From the foregoing, it will be appreciated that specific embodiments of the technology have been described herein for purposes of illustration, but that various modifications may be made without deviating from the disclosure. For example, the HIFU system 100 of FIG. 1 can include additional devices and/or systems to facilitate treatment or monitoring treatment of tissue. Certain aspects of the new technology described in the context of particular embodiments may be combined or eliminated in other embodiments. For example, mathematical models other than the Westervelt or KZK equations can be used to simplify nonlinear HIFU effects. In further embodiments, a look-up table can be associated with results from the Westervelt or other equation in addition to or in place of results from the KZK equation. Additionally, while advantages associated with certain embodiments of the new technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A method of calculating output of a high intensity focused ultrasound (HIFU) device, the method comprising:
treating a target site with a multi-element HIFU array, wherein the array comprises a generally spherical segment; and
simulating a field of the array by setting a boundary condition for the array, wherein setting a boundary condition comprises simplifying at least one geometrical aspect of the generally spherical segment.

2. The method of claim 1, further comprising:
using the boundary condition to substitute a single-element focused source for the array; and
applying a Westervelt equation to x, y, and z coordinates of the single-element focused source.

3. The method of claim 2 wherein using the boundary condition to substitute a single-element focused source for the array comprises setting an array aperture value, an array focal distance value, and an array pressure value of the single-element focused source.

4. The method of claim 1 wherein simulating a field of the array comprises applying a Rayleigh integral to determine the boundary condition.

5. The method of claim 1 wherein simulating a field of the array comprises applying a Khokhlov-Zabolotskaya-Kuznetsov (KZK) equation to x, y, and z coordinates of the generally spherical segment, and setting the boundary condition as a single-focused piston source.

6. The method of claim 5 wherein applying a KZK equation comprises using a look-up table to determine an aperture value and an initial pressure value corresponding to the single-focused piston source.

7. The method of claim 1 wherein simplifying at least one geometrical aspect of the generally spherical segment comprises modeling the generally spherical segment as a generally two-dimensional, circular segment.

8. The method of claim 1, further comprising numerically characterizing a pressure field created by radiation from the HIFU device.

9. A physical computer-readable storage medium having stored thereon, computer-executable instructions that, if executed by a computing system, cause the computing system to perform operations comprising:
treating a target site with a multi-element high intensity focused ultrasound (HIFU) array;
modeling the multi-element array as a single-element array, wherein modeling the multi-element array comprises setting a boundary condition for the multi-element array; and
gathering data related to the target site by simulating a field of the multi-element array using the single-element array.

10. The computer-readable storage medium of claim 9 wherein gathering data related to the target site comprises gathering data related to at least one of pressure, tissue condition, or biological effect at or proximate to tissue at the target site.

11. The computer-readable storage medium of claim 9 wherein modeling the multi-element array comprises applying a Westervelt equation to x, y, and z coordinates of the single-element array.

12. The computer-readable storage medium of claim 9 wherein the operations further comprise:
setting the boundary condition as a single-focused piston source; and
applying a Khokhlov-Zabolotskaya-Kuznetsov (KZK) equation to x, y, and z coordinates of the single-focused piston source.

13. The computer-readable storage medium of claim 12 wherein the operations further comprise using a look-up table to determine at least one of an aperture value or an initial pressure value corresponding to the single-focused piston source.

14. The computer-readable storage medium of claim 9 wherein treating a target site with a multi-element HIFU array comprises treating a target site in water, and wherein the operations further comprise extrapolating a result of the modeling to in situ conditions.

15. A method of characterizing nonlinear output of a high intensity focused ultrasound (HIFU) device, the method comprising:
modeling a three-dimensional geometric HIFU array as a two-dimensional array; and
applying a mathematical operation to the two-dimensional array, thereby simulating a field of the array, wherein applying a mathematical operation comprises characterizing at least one of a type or degree of nonlinear HIFU propagation.

16. The method of claim 15 wherein modeling a three-dimensional geometric array comprises modeling the array in water.

17. The method of claim 16, further comprising extrapolating a result of the mathematical operation to correspond to a HIFU treatment condition in situ.

18. The method of claim 15 wherein applying a mathematical operation to the two-dimensional array comprises applying a Westervelt equation to the two-dimensional array.

19. The method of claim 15 wherein applying a mathematical operation to the two-dimensional array comprises applying a Khokhlov-Zabolotskaya-Kuznetsov equation to the two-dimensional array.

20. The method of claim 15, further comprising populating a database with a value relating to a type or degree of nonlinear HIFU propagation effects for a particular HIFU source.
